(19) United States
(12) Patent Application Publication
(10) Pub. No.: US 2022/0387736 A1
(43) Pub. Date: Dec. 8, 2022

(54) NEBULIZER SYSTEM

(71) Applicant: MEDSHIELD, LLC, Loganville, GA (US)

(72) Inventors: Judit V. Barhite, Oxford, GA (US); Michael Crowley, Loganville, GA (US)

(21) Appl. No.: 17/364,514

(22) Filed: Jun. 30, 2021

Related U.S. Application Data
(60) Provisional application No. 63/093,247, filed on Oct. 18, 2020.

(51) Int. Cl. A61M 15/00

(52) U.S. Cl. CPC A61M 15/0018; A61M 15/009; A61M 15/0026; A61M 2205/7509; A61M 2205/7518

(57) ABSTRACT

The present invention is a nebulizer, aerosol, filter or inhalant handheld system. The device is composed of a non-invasive respiratory circuit with a medication reservoir that has an inlet that is configured to be connected to a gas source. In some embodiments, they include of one or more valves located downstream of the medication reservoir. Some embodiments include one or more filters, such as a bacterial viral filter located on the end of the exhalation port. The valve functions to prevent backflow of exhaled air and/or potential pathogens into the atmosphere. The filter functions to significantly reduce the dispersion of aerosols exhaled into the atmosphere and prevent further spread of potential pathogens. Some embodiment may include one or more pivotable elements allowing some portions of the nebulizer to pivot relative to other members.

### CROSS-REFERENCE TO RELATED APPLICATIONS

This application claims priority from co-pending U.S. Provisional Application Ser. No. 63/093,247 filed 18 Oct. 2020, which is titled “FILTERED NEBULIZER SYSTEM”, which is hereby incorporated in its entirety by reference.

### FIELD OF THE INVENTION

The present invention relates to apparatuses, methods and systems for generating an aerosol for delivery to a user/patient. More particularly, the present invention relates to filtered nebulizer, inhalant, aerosol, and/or valved handheld respiratory systems.

### BACKGROUND OF THE INVENTION

Various treatments, including aerosol therapy, have been a mainstay procedure for treating a multitude of pulmonary ailments, conditions, and diseases at home and in healthcare settings. The benefits of these therapies have been researched and proven to be superior to other therapies over the years. Nebulizers are used to administer medications, but, unfortunately, they have been shown to release ⅔ of the aerosol into the atmosphere. It has also been shown that these aerosols can stay airborne in the atmosphere for several hours. The unintentional release of these aerosols creates concern for health care workers and caregivers for the unintended inhalation of fugitive emissions during aerosol therapy, especially during pandemics (such as the Covid-19 coronavirus pandemic of 2020).

What is needed is a nebulizer which allows for the safe and effective administration of medication to a patient, while simultaneously, preventing the unintentional release of aerosols into the atmosphere.

### SUMMARY OF THE INVENTION

It is to be understood that both the foregoing general description and the following detailed description present embodiments of the invention, and are intended to provide an overview, or framework, for understanding the nature and character of the invention as it is claimed. The accompanying drawings are included to provide a further understanding of the invention, and are incorporated into and constitute part of this specification. The drawings illustrate various embodiments of the invention and, together with the description serve to explain the principles and operations of the invention.

The present invention is a nebulizer, aerosol, filter or inhalant handheld system. The improvement comprising of one or more valves and/or filters generally located downstream of the medication reservoir and/or in between the exhalation port and one or more filters. The valve can prevent backflow of exhaled air and/or potential pathogens into the atmosphere. The invention may also include one or more filters such as a bacterial viral filter located on the end of the exhalation port. The filter can significantly decrease the aerosols exhaled into the atmosphere and prevent further spread of potential pathogens. According to recent research, bacterial viral filters have an efficacy of 99.99%, thus an addition of a bacterial viral filter to various embodiments of the present invention significantly reduces the risk of unintended exhalation, and therefore the unintended inhalation of fugitive emissions during therapy by others.

In one embodiment, the invention is comprised of a body, a medication reservoir capable of holding one or more medications fluidly connected to the body, a mouthpiece fluidly connected to the body such that a user can inhale one or more medications from the medication reservoir, at least one exhaust port for allowing the user to exhale exhaust, one or more gas source inlets, and one or more valves.

In another embodiment, the device further comprises at least one filter in fluid communication with the exhaust.

In another embodiment, the device comprises a body, a medication reservoir capable of holding one or more medications fluidly connected to the body, a mouthpiece fluidly connected to the body such that a user can inhale one or more medications from the medication reservoir, at least one exhaust port for allowing the user to exhale exhaust, one or more gas source inlets, and one or more filters in fluid contact with the body.

In another embodiment, the device comprises a body, a medication reservoir capable of holding one or more medications fluidly connected to the body, a mouthpiece fluidly connected to the body such that a user can inhale one or more medications from the medication reservoir, at least one exhaust port for allowing the user to exhale exhaust, one or more gas source inlets, at least one valve, and at least one pivotable portion.

### BRIEF DESCRIPTION OF THE FIGURES

FIG. 1 is a frontal perspective view of an exemplary embodiment of the present invention;

FIG. 2 is a first side view of the exemplary embodiment of FIG. 1;

FIG. 3 is an opposite (second) side view of the exemplary embodiment of FIGS. 1-2;

FIG. 4 is a rear view of the exemplary embodiment of FIGS. 1-3;

FIG. 5 is a bottom view of the exemplary embodiment of FIGS. 1-4;

FIG. 6 is a top view of the exemplary embodiment of FIGS. 1-5;

FIG. 7 is a cut away view of the exemplary embodiment of FIG. 4 along section line A;

FIG. 8 is a cut away view of the exemplary embodiment of FIG. 3 along section line B;

FIG. 9 is an exploded view of the exemplary embodiment of FIGS. 1-8;

FIG. 10 is a frontal perspective view of another exemplary embodiment of the present invention;

FIG. 11 is a side view of the exemplary embodiment of FIG. 10;

FIG. 12 is a rear view of the exemplary embodiment of FIGS. 10-11;

FIG. 13 is an exploded view of the exemplary embodiment of FIGS. 10-12; and

FIG. 14 is a cut away view of the exemplary embodiment of FIGS. 10-13.

### DETAILED DESCRIPTION OF THE INVENTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains. In the figures, like reference numbers represent the same component.

The present invention is a nebulizer, aerosol, filter or inhalant handheld system. Various embodiments may include one or more valves or filters that may restrict or prevent the passage of specific gases/medications/particles/etc.

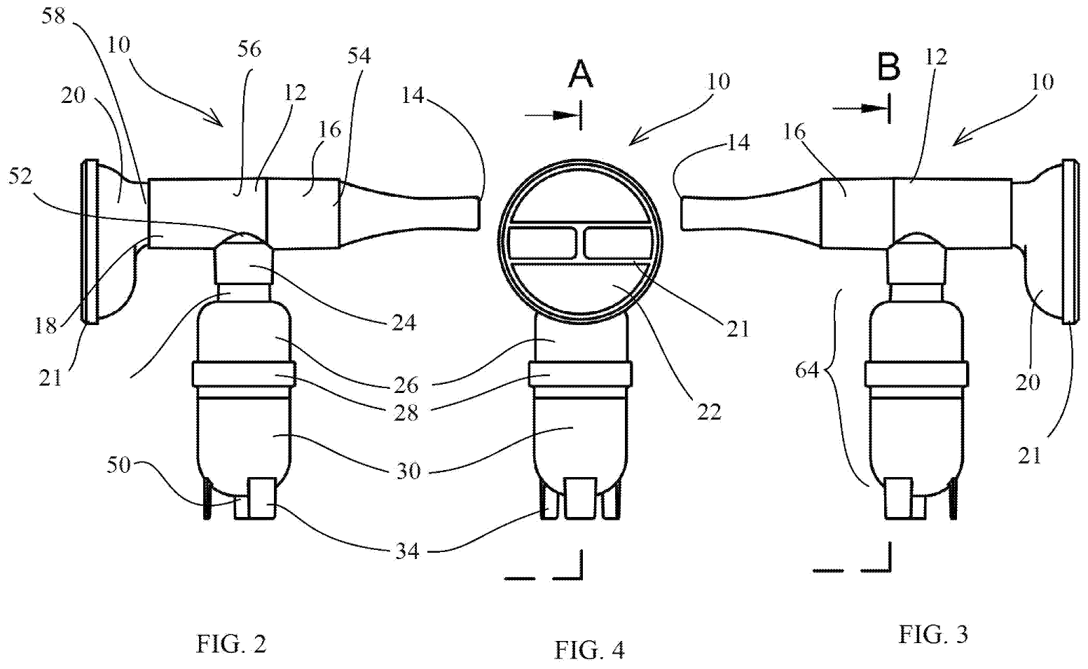

In some embodiments, one or more valves may be present. Valves may be selective, one-way, or two-way. In some embodiments there are one or more one-way valves that prevent the flow of the gas/fluid in the opposite direction. One-way valves may be located in between various components of the particular embodiment including, but not limited to, between (1) the gas source inlet and the medication reservoir(region 50, or in that approximate/proximal location, See FIG. 2), (2) the medication reservoir and the body/T-connector(region 52, or in that approximate/proximal location, See FIG. 2), (3) between the body/T-connector and the mouthpiece(region 54, or in that approximate/proximal location, See FIG. 2), (4) between the body/T-connector and the exhalation portion (which may be an open port, exhalation cap, other tubing, etc.) of the device(region 56, or in that approximate/proximal location, See FIGS. 2), and (5) in the body proximal the exhalation port/portion (region 58, or in that approximate/proximal location, See FIG. 2). One-way valves can be used to prevent backflow into other portions of the device (such as the medication reservoir, etc.). Note that although one-way valves are discussed in particular, other types of valves including selective, two-way, and others can be utilized in various embodiments and are within the scope of the present invention.

In some embodiments, one or more filters may be included. Such filters include any suitable filters such as bacteria viral filters. Filters may be located in between various components of the particular embodiment including, but not limited to, between (1) the gas source inlet and the medication reservoir (region 50, or in that approximate/proximal location, See FIG. 2), (2) the medication reservoir and the body/T-connector (region 52, or in that approximate/proximal location, See FIG. 2), (3) between the body/T-connector and the mouthpiece (region 54, or in that approximate/proximal location, See FIG. 2), (4) between the body/T-connector and the exhalation portion (which may be an open port, exhalation cap, other tubing, etc.) of the device (region 56, or in that approximate/proximal location, See FIGS. 2), and (5) in the body proximal the exhalation port/portion (region 58, or in that approximate/proximal location, See FIG. 2).

Some embodiments may include both one or more valves and one or more filters. In one embodiment, a valve can be located in between the exhalation port and a bacterial viral filter. This serves to direct exhaled aerosols out of the exhalation port and prevents backflow of exhaled aerosols back into the nebulizer system.

In some embodiments, a bacterial viral filter is located on the end of the exhalation port. The filter functions to significantly decrease the aerosols exhaled into the atmosphere and prevent further spread of potential pathogens.

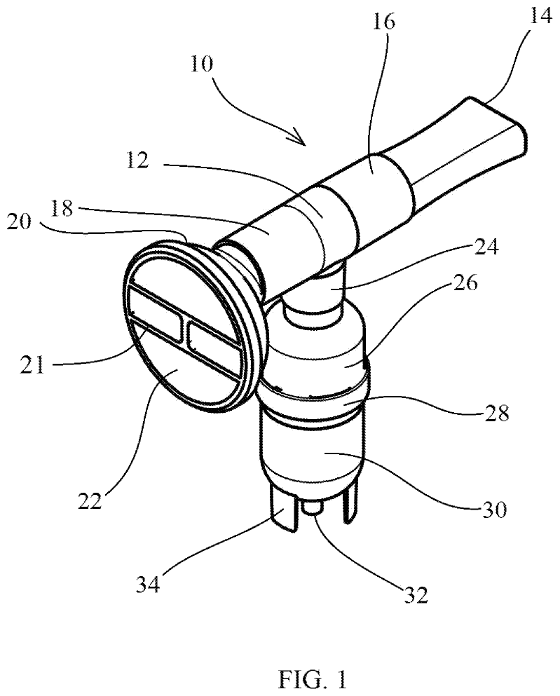

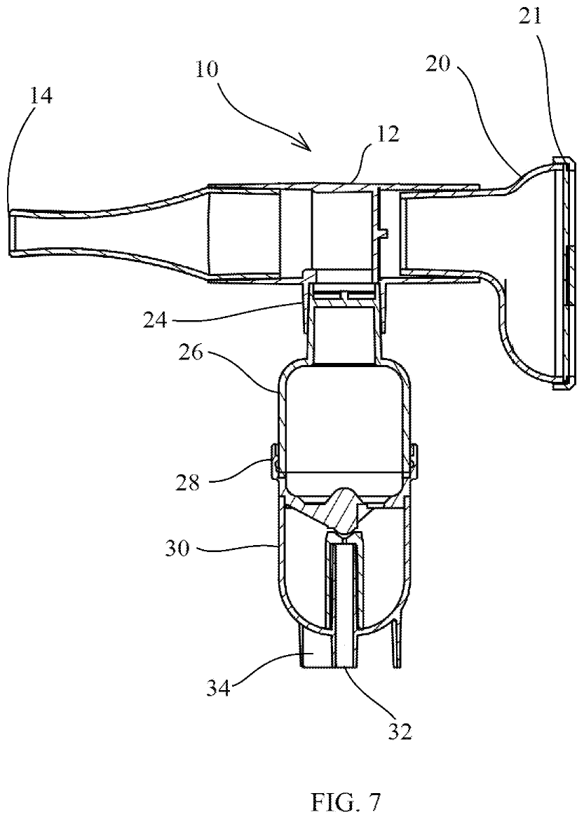

FIG. 1 illustrates an exemplary embodiment nebulizer system illustrating device/nebulizer 10, body/T-connecter/main body 12, mouthpiece 14, connector portion 16 of body/T-connector 12 (see FIG. 7), reservoir tube/exhalation portion 18 of body/T-connector 12 (see FIG. 7), exhalation cap 20, exhalation filter frame 21, exhalation filter/media 22, connector portion 24 of body/T-connector 12 (see FIG. 7), medication reservoir upper portion 26 (may have a valve inside in some embodiments (one way or flapper), connector 28, medication reservoir lower portion 30, gas source inlet/port 32, and supports 34 (not shown) which can be connected to a suitable fluid/gas/medication source.

Other elements not specifically designed but which may be included such as o-rings, gaskets, etc. between various members/elements, caps for various portions, and inserts (such as agitators). Various portions may be attached or connected to each other by any suitable means including, but not limited to: press fit, bonded, glued, welded, taped, etc. In addition, each portion/element may be broken down to/constructed of any number of subcomponents and the figures and description herein are only illustrative of a particular configuration. Similarly, multiple components may be constructed unitarily as desired.

FIG. 2 is a first side view of the exemplary embodiment of FIG. 1. Illustrated is device/nebulizer 10, body/T-connecter 12, mouthpiece 14, connector portion 16 of body/T-connector 12 (see FIG. 7), reservoir tube/exhalation portion 18 of body/T-connector 12 (see FIG. 7), exhalation cap 20, exhalation filter frame 21, exhalation filter/media 22, connector portion 24 of body/T-connector 12 (see FIG. 7), medication reservoir upper portion 26, connector 28, medication reservoir lower portion 30, and gas source inlet/port 32, and supports 34. Regions 50, 52, 54, 56 and 58 represent particular regions in which a valve and/or filter and/or pivotable element may be positioned (note that these designate approximate areas and the exact location can vary).

FIG. 3 is an opposite (second) side view of the exemplary embodiment of FIGS. 1-2. Illustrated are many of the same features/elements/portions of FIGS. 1-2 wherein like numbers reference like portions/elements/features (similarly throughout each of the figures in the figures collectively). Also designated particularly in this figure are medical portion/medicinal portion/channel 64. Medical portion 64 can be defined as including the medication reservoir 26/30 and any connectors, agitators, etc. between the gas inlet and the body.

FIG. 4 is a rear view of the exemplary embodiment of FIGS. 1-3 illustrating an exemplary arraignment of various elements of the embodiment.

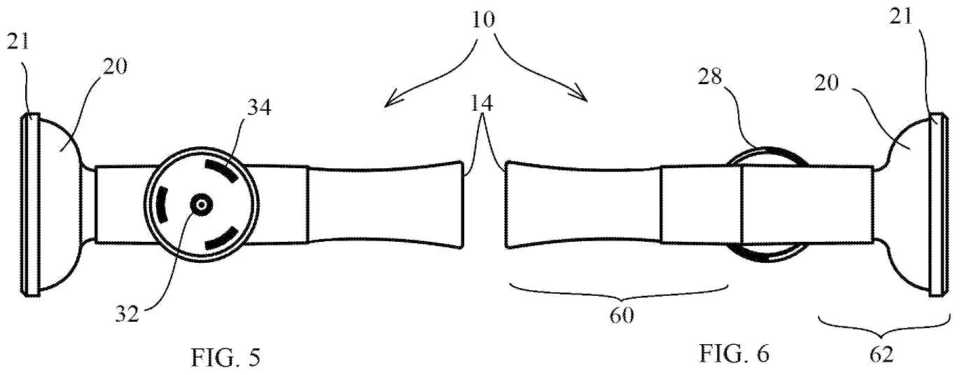

FIG. 5 is a bottom view of the exemplary embodiment of FIGS. 1-4 illustrating an exemplary arraignment of various elements of the embodiment.

FIG. 6 is a top view of the exemplary embodiment of FIGS. 1-5 illustrating an exemplary arraignment of various elements of the embodiment. Note that inhalation portion/channel 60 is shown as an approximate location and may extend farther as appropriate. This channel is defined as the flow path that gas/fluids/medication takes when being inhaled as opposed to the medical portion/medicinal portion/channel 64 (See FIG. 3) and exhalation portion/channel 62. In general, the channels 60, 62, 64 are the particular flow path of gas/medicine/etc. As should be understood, there is a continuity between the medical, inhalation and exhalation portions such that the gas/medicine/etc. is in one of the three portions while inside device 10.

FIG. 7 is a cut away view of the exemplary embodiment of FIG. 4 along section line A illustrating an exemplary arraignment of various elements of the embodiment.

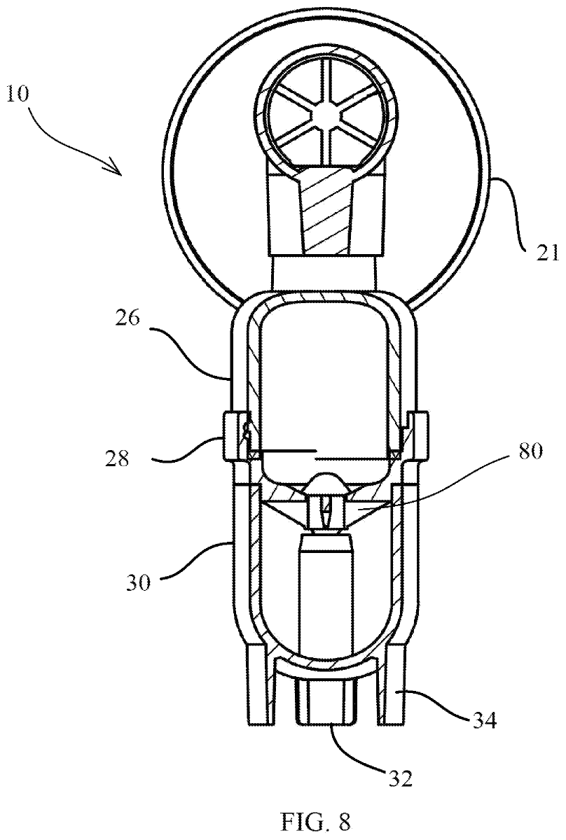

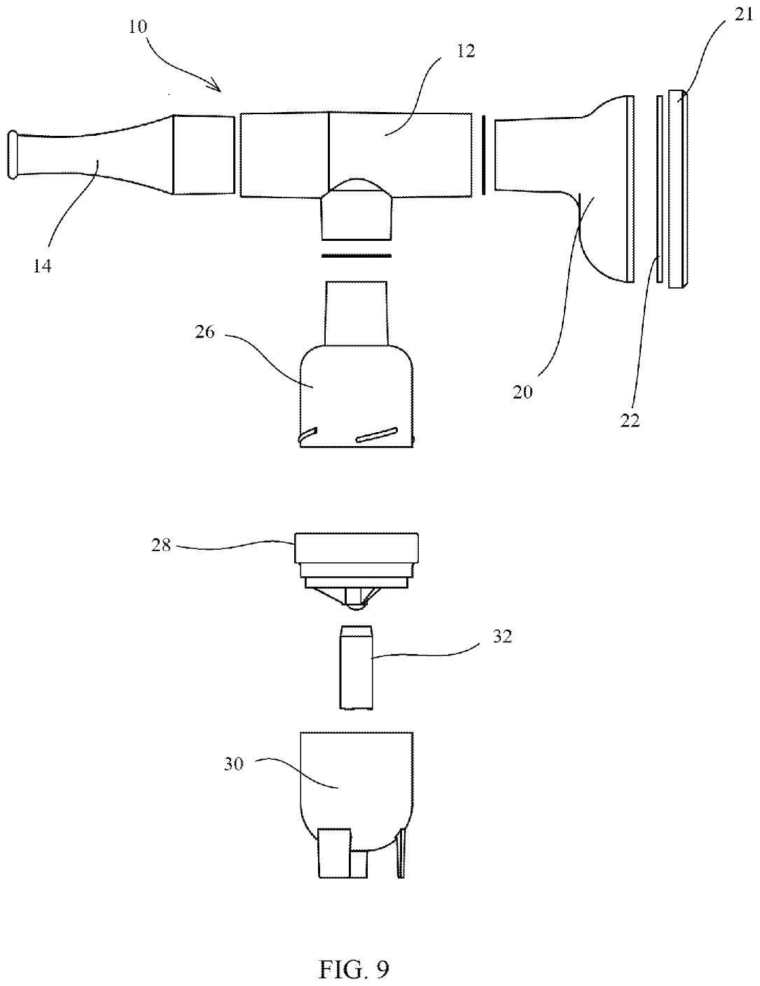

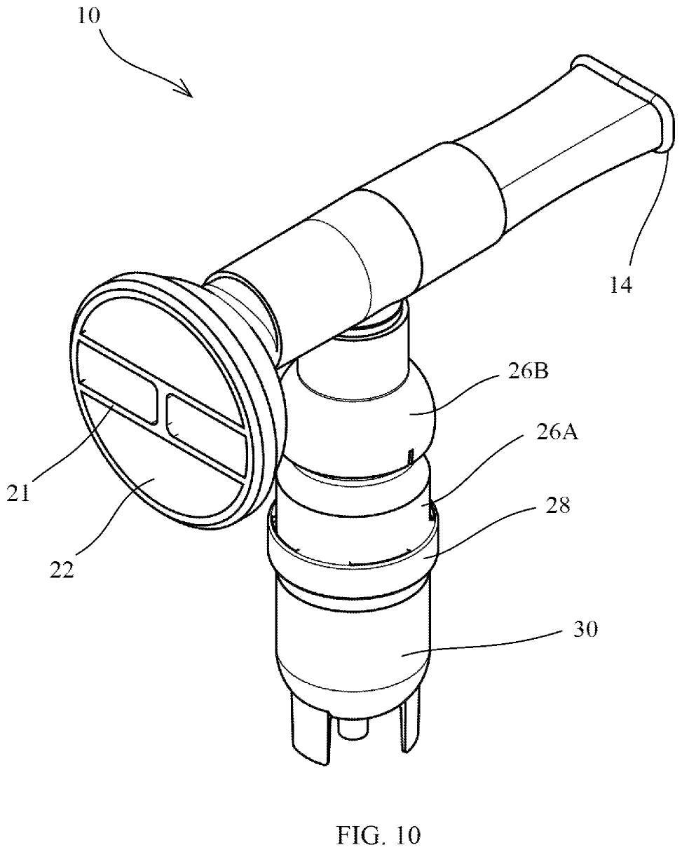

FIG. 8 is a cut away view of the exemplary embodiment of FIG. 3 along section line B illustrating an exemplary arraignment of various elements of the embodiment. Also illustrated in this illustration are some inner chambers of an exemplary embodiment as well as agitator 80 for agitating or mixing of the medicine/gas/etc.

FIG. 9 is an exploded view of the exemplary embodiment of FIGS. 1-8 illustrating an exemplary arraignment of various elements of the embodiment.

As one in the medical field can appreciate/understand, medicine is placed into the medicine reservoir so that it can be aerosolized and breathed in by a user/patient. It can be desirable for the medicine to be maintained in the medicine reservoir in a predetermined orientation with respect to the reservoir to maximize/optimize aerosol-ability. For example, in some instances, it is desirable for the medicine to be maintained in a horizontal fashion. There is a need for a device in which the medicine reservoir (and thus the medicine it contains) can be maintained in a predetermined orientation even if other parts/components/portions of the nebulizer are rotated/pivoted/reoriented with respect to the medicine reservoir. Thus, in some embodiments of the present invention, upper portions of the nebulizer are able to be pivoted relative to the medicine reservoir. FIGS. 10-14 illustrate an exemplary pivotable nebulizer system.

FIG. 10 is a frontal perspective view of another exemplary embodiment of the present invention. In this embodiment, one or more pivotable elements/portions are included in nebulizer/device 10. This embodiment illustrates nebulizer in which medication reservoir upper portion 26 is constructed as two components, portion 26A and 26B. In this embodiment, one or more pivotable elements/portions form a rounded portion (26B/26A) that mates pairs with another component (i.e., 26A/26B) of device 10 together form a ball and socket type of joint which allows/permits medication reservoir 26/30 to be able to be maintained in one orientation (such as the portion is vertical/the medicine is horizontal, relatively), even if body 12 is rotated relative to it (due to the design of the valve, it can be constructed to minimize or eliminate leakage of the medicine/gas/etc.). Thus, even if body 12 is rotated relative to medication reservoir 26/30, the medicine is in the proper orientation to be properly aerosolized to be inhaled by the user whereas if medication reservoir 26/30 itself were pivoted, the medicine in it may no longer be in the ideal orientation to be aerosolized to be inhaled (this could result in the patient getting an improper dose of the medication, or even none at all).

Note that other types of pivotable mechanisms may be utilized in place of the ball and socket pairing. So long as the medicine can be maintained in a predetermined orientation even if other portions of the nebulizer are pivoted, then it would be an acceptable substitution in some embodiments (care should be taken, of course, to minimize or eliminate leakage). In this embodiment, the pivotable element is part of the medicine reservoir, but this is not strictly required. So long as the pivot is at, or above the medicine level, other embodiments may include other pivotable elements. In some embodiments, one or more pivotable elements may be located between the medicine of the medicine reservoir and body 12. Other embodiments may also include additional pivotable elements such as in the inhalation portion, exhalation portion, or body portion. In some embodiments there is at least one pivotable element proximate the medicine reservoir. In some embodiments there is at least one pivotable element between the medicine reservoir (or unitary therewith) and body 12. In another embodiment, there is at least one pivotable element between the medicine reservoir (or unitary therewith) and before body 12. In another embodiment, the device includes at least one valve located above/downstream/proximate the medication reservoir and at least one pivotable element is positioned/located between the valve and the body. In another embodiment, the device includes at least one valve located above/downstream/proximate the medication reservoir and at least one pivotable element is positioned/located between the medication reservoir and the body. Each embodiment of the present invention has at least one of the following: (A) one or more valves, (B) one or more filters, and (C) one or more pivotable elements. Various combinations of these are in various embodiments of the present invention.

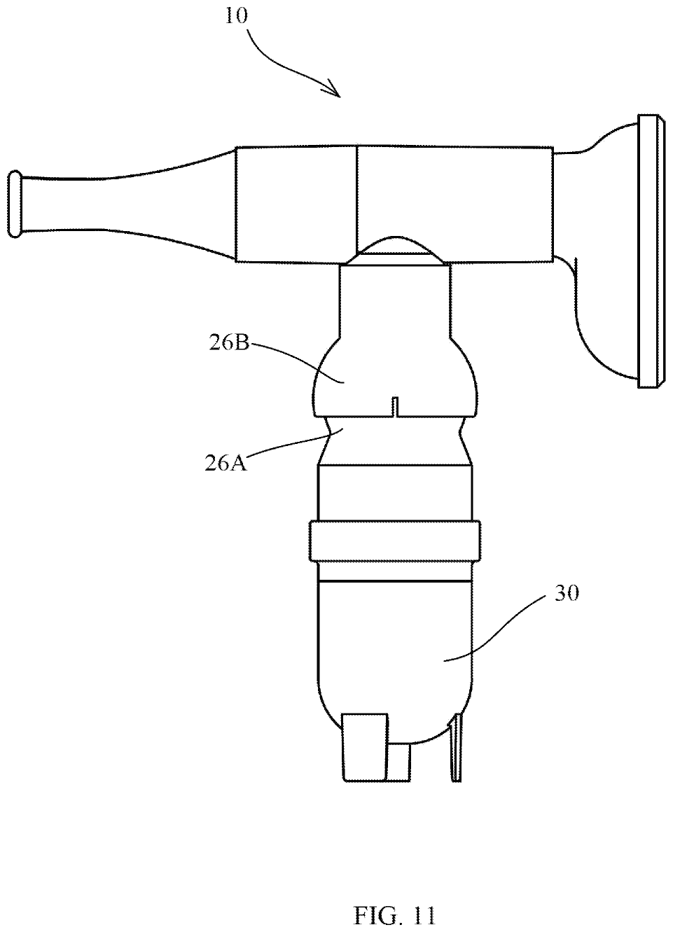

FIG. 11 is a side view of the exemplary embodiment of FIG. 10 illustrating an exemplary arraignment of various elements of the embodiment.

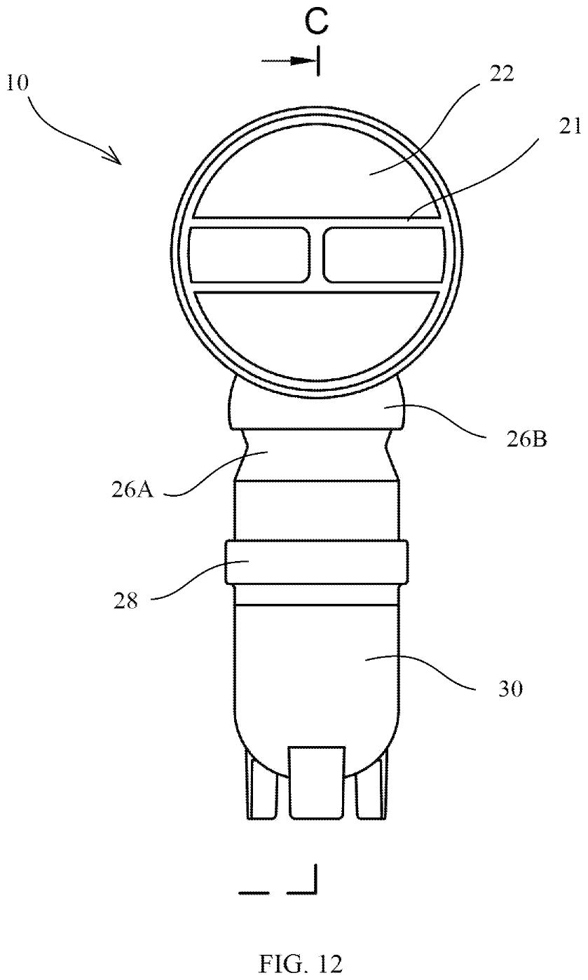

FIG. 12 is a rear view of the exemplary embodiment of FIGS. 10-11 illustrating an exemplary arraignment of various elements of the embodiment.

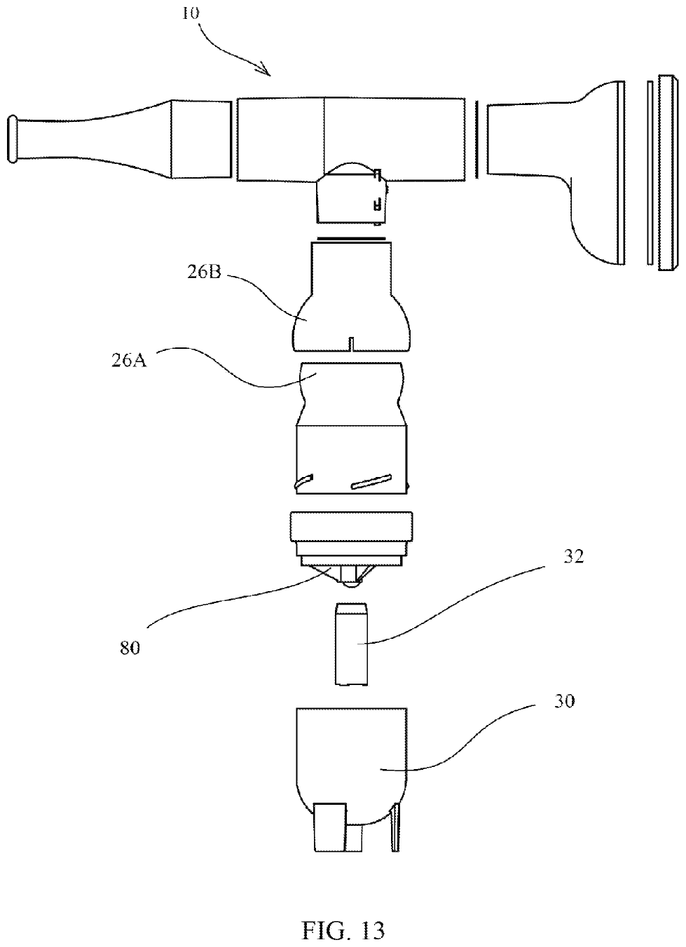

FIG. 13 is an exploded view of the exemplary embodiment of FIGS. 10-12 illustrating an exemplary arraignment of various elements of the embodiment. Note that this view illustrates a particular arrangement for portions 26A and 26B to allow the medical reservoir to pivot relative to some other portions of device 10.

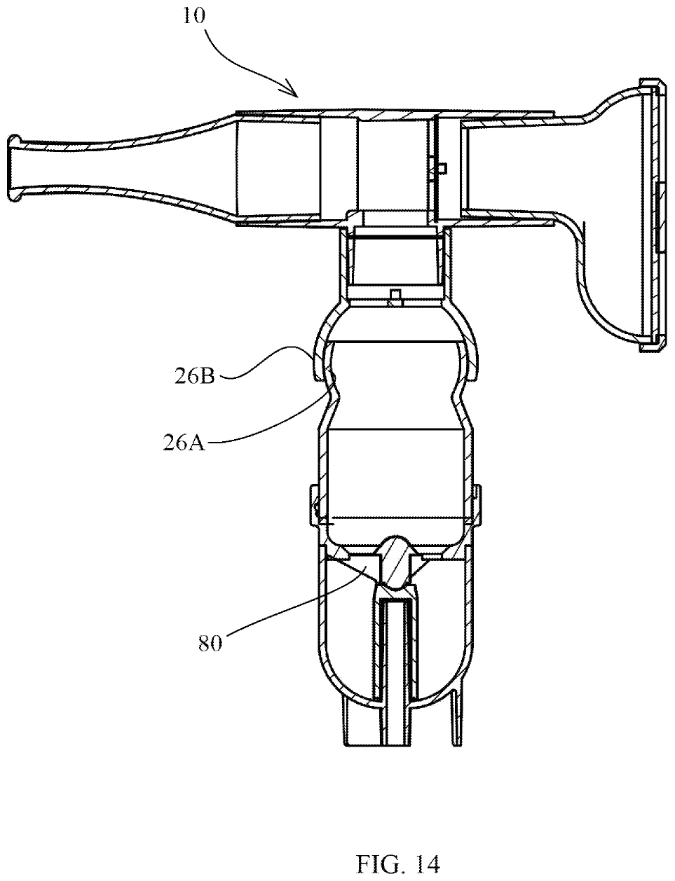

FIG. 14 is a cut away view of the exemplary embodiment of FIGS. 10-13 along section line C of FIG. 12 illustrating an exemplary arraignment of various elements of the embodiment.

As described herein in some embodiments may include one or more pivotable elements/portions to provide/assist in the consistent supply of aerosolized medicine

In the healthcare setting, aerosol therapy is typically administered in an emergency or urgent setting. One of the necessities for this type of therapy is ease of use of the equipment to administer these medications and being able to obtain the equipment efficiently.

Although various components of the present invention may be illustrated as being of a particular shape for convenience, such components may be of any suitable shape, configuration, orientation, etc.

While the specification has been described in detail with respect to specific embodiments thereof, it will be appreciated that those skilled in the art, upon attaining an understanding of the foregoing, may readily conceive of alterations to, variations of, and equivalents to these embodiments.

What is claimed is:

1. A nebulizer system, said system comprised of:
a body,
a medication reservoir capable of holding one or more medications fluidly connected to said body,
a mouthpiece fluidly connected to said body such that a user can inhale said one or more medications from said medication reservoir,
at least one exhaust port for allowing said user to exhale exhaust,
one or more gas source inlets, and
one or more valves.

2. The nebulizer system of claim 1, further comprising at least one filter in fluid communication with said body.

3. The nebulizer system of claim 1, wherein said at least one valve is a one-way valve and said at least one valve is located between said gas source inlet and said medication reservoir.

4. The nebulizer system of claim 1, wherein said at least one valve is located between said medication reservoir and said body.

5. The nebulizer system of claim 1, wherein said at least one valve is located between said body and said mouthpiece.

6. The nebulizer system of claim 1, wherein said at least one valve is located between said body and said exhalation port.

7. The nebulizer system of claim 1, further comprising at least one bacterial viral filter located downstream of said body.

8. The nebulizer system of claim 1, further comprising at least one pivotable portion.

9. The nebulizer system of claim 1, further comprising at least one pivotable portion wherein said medication reservoir is able to pivot relative to said mouthpiece due to said at least one pivotable portion.

10. A nebulizer system, said system comprised of:
a body,
a medication reservoir capable of holding one or more medications fluidly connected to said body,
a mouthpiece fluidly connected to said body such that a user can inhale said one or more medications from said medication reservoir,
at least one exhaust port for allowing said user to exhale exhaust,
one or more gas source inlets, and
one or more filters in fluid contact with said body.

11. The nebulizer system of claim 10, further comprising at least one valve in fluid communication with said body.

12. The nebulizer system of claim 10, wherein said at least one filter is located between said gas source inlet and said medication reservoir.

13. The nebulizer system of claim 10, wherein said at least one filter is located between said medication reservoir and said body.

14. The nebulizer system of claim 10, wherein said at least one filter is located between said body and said mouthpiece.

15. The nebulizer system of claim 10, wherein said at least one filter is located between said body and said exhalation port.

16. The nebulizer system of claim 10, further comprising at least one bacterial viral filter located proximate said exhalation port.

17. The nebulizer system of claim 10, further comprising at least one pivotable portion.

18. The nebulizer system of claim 10, further comprising at least one pivotable portion wherein said medication reservoir is able to pivot relative to said mouthpiece due to said at least one pivotable portion.

19. An nebulizer system, said system comprised of:
a body,
a medication reservoir capable of holding one or more medications fluidly connected to said body,
a mouthpiece fluidly connected to said body such that a user can inhale said one or more medications from said medication reservoir,
at least one exhaust port for allowing said user to exhale exhaust,
one or more gas source inlets,
at least one valve, and
at least one pivotable portion.

20. The nebulizer system of claim 19, further comprising one or more filters in fluid contact with said body.
